(19) United States
(12) Patent Application Publication
(10) Pub. No.: US 2026/0265655 A1
(43) Pub. Date: Sep. 10, 2026

(54) ANTIMICROBIAL SURFACES AND USES THEREOF IN CELL CULTURES

(71) Applicant: THE CULTIVATED B. GMBH, Heidelberg (DE)

(72) Inventor: Hamid R. NOORI, Heidelberg (DE)

(21) Appl. No.: 19/490,077

(22) PCT Filed: Jun. 6, 2024
(86) PCT No.: PCT/EP2024/065557

§ 371 Date: Dec. 4, 2025

(30) Foreign Application Priority Data
Jun. 7, 2023 (EP) 23177906.7

(51) Int. Cl. C12M 1/00; C12M 1/12

(52) U.S. Cl. CPC C12M 23/20; C12M 25/16

(57) ABSTRACT

The present invention relates to a cell culture vessel characterized in that it comprises an inner surface on which one or more lectins suitable for binding to one or more species of microorganisms are immobilized. Furthermore, the invention refers to a method for cultivating cells comprising contacting the cell culture medium comprising the cells with a surface that bears immobilized lectin and to the use of immobilized lectin for reducing the microorganism contamination in a cell culture contacted with such surface.

The present invention relates to a cell culture vessel characterized in that it comprises an inner surface on which one or more lectins suitable for binding to one or more species of microorganisms are immobilized. Furthermore, the invention refers to a method for cultivating cells comprising contacting the cell culture medium comprising the cells with a surface that bears immobilized lectin and to the use of immobilized lectin for reducing the microorganism contamination in a cell culture contacted with such surface.

Cell cultures are widely used for various purposes. For decades, cell cultures were used for research purposes. For example, cells are used to investigate the effects of chemical agents, hormones, medicaments, pharmaceutical candidates, toxins and mixtures thereof. Further, effects of other external influences such as cell culture media, effects of pathogens (also: germs) such a viruses, bacteria and protozoa, of temperature, pH, irradiations, etc. are investigated. Moreover, intracellular processes are investigated as well as differentiation, de-differentiation, mutagenesis, neoplastic progression of cells. The interaction of different cell types and even cells of different species are observable in cell cultures. In addition to such investigative studies, cell cultures are described for preparing material. For instance, cell cultures are used for the homologous or heterologous expression of polypeptides. In this context, cell cultures are usable for preparing antibodies such as monoclonal and even humanized monoclonal antibodies, of hormones, or other proteins. Cell cultures are described for preparation of non-peptidic compounds such as small-molecular sugars, polysaccharides, lipids, vitamins, etc. A newer development is the consideration to use cell cultures for preparing comestible material. For instance, cell cultures may be used for preparing cultivated meat, which may include alternative proteins via precision fermentation, and production of biologic drugs as potential use cases of cell cultures. There are numerous further application fields/domains of cell cultures.

A critical issue regarding the use of cell cultures is the often-occurring microbial contamination, in particular contamination with bacteria such as Mycoplasma species. Though it is typically worked with high care to avoid contaminations, it cannot be guaranteed that microbes do not enter the cell culture vessel.

Especially, Mycoplasma species are often considered of concern. Mycoplasma species have a particularly small size of often not more than 3 μm and are thus hardly removable from cell culture media by filtering, centrifugation etc. In particular, bioreactors used for animal cell lines often need to be integrated in a cleanroom environment since the systems are susceptible to contamination by unwanted microorganism such as Mycoplasma species. For larger size industrial applications, particularly food manufacturing, cleanrooms are not or only hardly a feasible infrastructure due to high capital and operative expenditures associated with them.

In scientific context, such contamination is often prevented by addition of antibiotics such as penicillins, cephalosporins, aminoglycosides, tetracyclines, macrolides or fluoroquinolones to the cell culture medium. This has, however, significant drawbacks. It can have undesired influences on the cellular behavior. When preparing a pharmaceutical or cosmetic material or even a comestible good from the cell culture the presence of residual antibiotic is even less desirable. In fact, many antibiotics may lead to an undesired antibiotic resistance formation when applied multiple times in moderate or low doses. Furthermore, proliferation rates of microorganisms that are already antibiotic resistant are particularly difficult to control or hamper, even in the presence of antibiotics. Accordingly, there is a need for means for cell cultures that allow omittance of antibiotics and still keep the microbial contamination low.

Anjana and Anitha (Int. J. Curr. Sci., 2012, 17-23) describe silver nitrate nanoparticles mixed with lectin and that these nanoparticles have an antifungal effect in suspension which is usable in an agronomic context of fighting rice pathogen. This method has the significant drawback that a cell culture would be contaminated with the nanoparticles which would be hardly and not readily removable and may have undesired side effects. There is still the need for means that allow widely omittance of agents that are present in the cell suspension or the product and still keep the microbial contamination low.

In contrast to medical devices that are contacted with or even implanted in a patient's body, cell cultures do not comprise an immune system and the cell culture medium typically remains a limited volume contacted with the cells for a longer time. Furthermore, cell culture vessels typically have an inner surface that is rather hard and smooth to avoid bacterial attachment such as stainless steel, glass, polystyrene or polycarbonate, whereas medical devices such as catheters are rather flexible.

A focus of coating medical devices such as catheters seems to lay on coating with silver salts and colloidal silver. In this context, Gu et al. (World Journal of Microbiology & Biotechnology, 2001, 17:173-179) described that catheter surface can be immersed with silver salts and lectins and subsequently dried. WO 2004/017738 teaches the use of colloidal metals such as silver for coating surfaces. Such coated surfaces do however not seem to be readily suitable for cell culture. WO 2014/138885 teaches the use of silver component mixed with a polymer. Riau et al. (Pathogens, 2019, 8(93)) teaches the use of nano-silver and release of silver therefrom to achieve an antimicrobial effect on medical devices based on the release of silver salts. The antimicrobial agents of Gu et al., WO 2004/017738, WO 2014/138885 or Riau et al. are not immobilized on the surface and are thus rather easily washed during cell culture process. Furthermore, the catheter as a whole is immersed during the coating process. In contrast to a flexible catheter, a typical cell culture vessel is made of materials such as stainless steel, glass, polystyrene or polycarbonate are less flexible, have a smoother surface and bear even lower adherence of antimicrobial agents. In addition, due to their size, cell culture vessels as described for the present invention cannot reasonably be completely immersed in a coating solution. All these characteristics necessitate alternative methods than those described above.

WO 2013/012924 describes engineered microbe-targeting molecules that can be attached to the surface of microbeads mainly used for diagnostic purposes. Here, a lectin-based fusion protein is prepared. WO 2013/012924 does not describe the purposeful use of means for improving cell cultures.

There is still the need for means for reducing microbial contamination in cell cultures without need of antibiotics.

Surprisingly, it has been found that contacting the cell culture medium comprising the cells of interest with a surface on which one or more lectins suitable for binding to one or more species of microorganisms are immobilized leads to the reduction of microbial contamination in the cell culture without need of antibiotics.

A first aspect of the invention relates to a cell culture vessel characterized in that it comprises an inner surface on which one or more lectins suitable for binding to one or more species of microorganisms are immobilized.

In other words, the present invention also refers to a cell culture vessel characterized in that the inner surface bears one or more lectins suitable for binding to one or more species of microorganisms.

The cell culture vessel may be suitable for any cell culture. In a preferred embodiment, it is suitable for culturing eukaryotic cells. In a preferred embodiment, it is suitable for culturing human, animal, fungal or plant cells. In a preferred embodiment, it is suitable for culturing human or animal cells, in particular human or other mammalian cells. In a preferred embodiment, the cell culture vessel is suitable for preparing comestible goods.

The inner surface of the cell culture vessel may also be understood as the inner side or as the interior or the inner wall of the cell culture vessel or the inner wall of the cell culture vessel. The inner surface of the cell culture vessel may be considered as antimicrobial or antiseptic surface.

In a preferred embodiment, binding of the one or more lectins to the one or more species of microorganisms is non-covalent binding.

As used throughout the present invention, “suitable for binding” may be understood in the broadest sense. In a preferred embodiment, “suitable for binding” means binding with a dissociation constant (Kd) of less than 10 μM, less than 1 μM, less than 500 nM, less than 200 nM, less than 100 nM, or less than 50 nM.

In a preferred embodiment, binding of the one or more lectins to the one or more species of microorganisms is specific binding. As used throughout the present invention, “specific binding” or “specifically binding” may be understood in the broadest sense as generally understood in the art. Preferably, “specific binding” or “specifically binding” indicates that binding of the lectin to the designated saccharide target has an at least 1.5 fold, preferably at least 2 fold, more preferably at least 5 fold, in particular preferably at least 10 fold or even 100 fold higher affinity than to a random saccharide such as, e.g., starch or sepharose.

A lectin suitable for binding to one or more species of microorganisms may be any lectin that is able to bind a surficial structure or molecular moiety (in particular a saccharide moiety of at least one microorganism. A lectin may be understood in the broadest sense as generally understood in the art. A lectin may be a carbohydrate-binding protein that is specific for one or more saccharide moieties that may be conjugated to other structures such as proteins (i.e., forming part of a glycoprotein) or lipids (i.e., forming part of a glycolipid), often on the surface of microorganisms. Soluble lectins may lead to agglutination of microorganisms that bear such one or more saccharide moieties on their surface. Many lectins have a role in recognition at the cellular and molecular level and may further play a role in biological recognition of microorganisms or other cells. Lectins are known to be able to interact with microorganisms such as bacteria, viruses and/or fungi. In a preferred embodiment, a lectin used in the context of the present invention does (essentially) not possess enzymatic activity, in particular does (essentially) not possess enzymatic activity digesting its target carbohydrate.

As used herein, the terms “saccharide moiety” and “sugar moiety” are understood interchangeably in the broadest sense as any saccharide that is presented by a microorganism and may be bound. Typically, such saccharide moiety is located at the outer surface of a microorganism. Preferably, a saccharide moiety comprises: one or more hydroxy groups (also: hydroxyl groups, —OH); and/or one or more ether groups (—O—) and/or one or more aldehyde or ketone groups (═O).

Optionally, a saccharide moiety may further comprise one or more other functional groups such as one or more carboxy groups (—COOH) or salts thereof (—COO⁺+cation(s) as counterion(s)), one or more primary amine groups (—NH₂) or salts thereof (—NH₃⁺+anion(s) as counterion(s)), one or more secondary amine groups (—NH—), one or more amide groups (—NH—CO—, for example —NH—CO—CH₃ residues), and/or one or more functional groups containing sulfur and/or phosphor. A saccharide moiety may also be a carbohydrate moiety. A saccharide moiety may be a monosaccharide moiety, a disaccharide moiety, an oligosaccharide moiety, or a polysaccharide moiety. A saccharide moiety having three or more saccharide units may be branched or unbranched. It will be understood that the term “saccharide moiety” may also include salts and modified forms thereof.

As used herein, the terms “protein” and “polypeptide” may be understood interchangeably in the broadest sense as a compound mainly composed of natural amino acid moieties consecutively conjugated with another via amide bonds. It will be understood that a protein in the sense of the present invention may or may not be subjected to one or more posttranslational modification(s) and/or be conjugated with one or more non-amino acid moieties. For example, one or more of the termini of the protein may or may not be capped by any means known in the art, such as, e.g., amidation, acetylation, methylation, acylation. Optional posttranslational modifications are well-known in the art and may be but may not be limited to disulfide bond, phosphorylation, amidation, sulfatation, glycosylation, truncation, oxidation, reduction, lipidation, decarboxylation, acetylation, deamidation, formation, amino acid addition, cofactor addition (e.g., biotinylation, heme addition, eicosanoid addition, steroid addition) and complexation of metal ions, non-metal ions, peptides or small molecules and addition of iron-sulphide clusters. It will be understood that such protein may also bear one or more non-natural amino acid moiety/moieties and/or one or more posttranscriptional modification(s) and/or may be conjugated to one or more further structures such as label moieties (e.g., by means of a dye (e.g., a fluorescence dye) or a metal label (e.g., gold beads)).

In a preferred embodiment, the one or more lectins are selected from the group consisting of C-type lectins, a fragment suitable for binding to one or more species of microorganisms thereof, and a fusion protein comprising a C-type lectin or a fragment suitable for binding to one or more species of microorganisms thereof.

As throughout the present invention, a fragment of a lectin may be any truncated form of a naturally occurring lectin that still specifically binds one or more saccharide moieties of the one or more microorganisms of interest. As used herein, truncated may mean for example truncation by one, two, three, up to five, up to ten, up to 20, up to 50 or up to 100 amino acid moieties or more than 100 amino acid moieties of the respected naturally occurring lectin.

As throughout the present invention, a fusion protein comprising a lectin is any protein that comprises a lectin and one or more further consecutive amino acid moieties, five or more consecutive amino acid moieties, ten or more consecutive amino acid moieties, or twenty or more consecutive amino acid moieties.

As throughout the present invention, a fusion protein comprising a lectin fragment is any protein that comprises a lectin fragment and one or more further consecutive amino acid moieties, five or more consecutive amino acid moieties, ten or more consecutive amino acid moieties, or twenty or more consecutive amino acid moieties.

In a preferred embodiment, the one or more lectins are selected from the group consisting of C-type lectins. Such C-type lectins as such and their effect in human bodies are known such as from Mnich et al., Front. Cell. Infect. Microbiol., 20220, 10:309, doi: 10.3389/fcimb.2020.00309).

In a preferred embodiment, the one or more lectins are selected from the group consisting of collectins, a fragment suitable for binding to one or more species of microorganisms thereof, and a fusion protein comprising a collectin or a fragment suitable for binding to one or more species of microorganisms thereof. In a preferred embodiment, the one or more lectins are selected from the group consisting of collectins.

In a preferred embodiment, the one or more lectins are selected from the group consisting of surfactant protein A (SP-A), surfactant protein D (SP-D), mannose-binding lectin (MBL), collectin liver 1 (CL-L1), collectin placenta 1 (CL-P1), collectin kidney 1 (CL-K1), conglutinin, collectin of 43 kDa (CL-43), collectin of 46 kDa (CL-46), a fragment suitable for binding to one or more species of microorganisms thereof, and a fusion protein comprising at least one thereof.

The person skilled in the art knows such lectins as such. A number of such lectins and their effects in higher organisms is for instance described in Liu et al., Molecules 2015, 20:2272-2295.

In a preferred embodiment, the one or more lectins are selected from the group consisting of surfactant protein A (SP-A), surfactant protein D (SP-D), mannose-binding lectin (MBL), collectin liver 1 (CL-L1), collectin placenta 1 (CL-P1), collectin kidney 1 (CL-K1), conglutinin, collectin of 43 kDa (CL-43), and collectin of 46 kDa (CL-46). In a preferred embodiment, the one or more lectins are selected from the group consisting of surfactant protein A (SP-A), surfactant protein D (SP-D), and mannose-binding lectin (MBL), a fragment suitable for binding to one or more species of microorganisms thereof, and a fusion protein comprising at least one thereof. In a preferred embodiment, the one or more lectins are selected from the group consisting of surfactant protein A (SP-A), surfactant protein D (SP-D), and mannose-binding lectin (MBL).

In a preferred embodiment, the one or more lectins comprise or consist of surfactant protein A, a fragment suitable for binding to one or more species of microorganisms thereof, and a fusion protein comprising at least one thereof. In a preferred embodiment, the one or more lectins comprise or consist of surfactant protein A or a fusion protein comprising surfactant protein A (SP-A). SP-A as such is found in multicellular organisms such as human is known by the person skilled in the art such as Piboonpocanun et al., The Journal of Biological Chemistry, 2005, 280(1):9-17).

As used herein, a microorganism may be understood in the broadest sense as any organism of microscopic size. The terms “microorganism” and “microbe” may be understood interchangeably. A microorganism may be in its single-celled form or as a colony of cells or a polycellular organism of microscopic size. As used herein, microscopic size may be a diameter of less than 100 μm, less than 10 μm, or even less than 1 μm. A microorganism may be a prokaryote (e.g., a bacterial cell or an archaebacterial cell) or a eukaryote (e.g., a protist cell, a fungal cell or an animal cell).

In a preferred embodiment, the one or more species of microorganisms are selected from the group consisting of bacteria, protists or a combination thereof. In a preferred embodiment, the one or more species of microorganisms are selected from the group consisting of infectious cells, in particular human pathogenic cells. In a preferred embodiment, the one or more species of microorganisms are selected from bacteria, in particular human pathogenic bacteria. In a preferred embodiment, the one or more species of microorganisms are selected from the group consisting of mollicutes. In a preferred embodiment, the one or more species of microorganisms are selected from the group consisting of mycoplasmataceae. In a preferred embodiment, the one or more species of microorganisms are selected from the group consisting of Mycoplasma species. Mycoplasma species may be any Mycoplasma species. For instance, it is a Mycoplasma species selected from the group consisting of Mycoplasma pneumonia, Mycoplasma amphoriforme, Mycoplasma buccale, Mycoplasma faucium, Mycoplasma fermentans, Mycoplasma genitalium, Mycoplasma hominis, Mycoplasma incognitus, Mycoplasma lipophilum, Mycoplasma orale, Mycoplasma penetrans, Mycoplasma pirum, Mycoplasma primatum, Mycoplasma salivarium, and Mycoplasma spermatophilum.

The cell culture may be any container suitable for cell culture. A cell culture vessel may be of any size and material suitable for such purpose. A cell culture vessel may be large size container or may be a cell culture flask.

A cell culture vessel, in particular when it is a large size container, may also be designated as “bioreactor”.

A large size container may optionally be equipped with one or more inlets and/or outlets (e.g. for gas in- and outflow, medium in- and outflow, withdrawal of cells, etc.), with one or more stirrers, etc. A cell culture vessel may have a volume of below 5 mL, between 5 mL and 100 mL, between 100 mL and 1 L, between 1 L and 100 L, between 100 L and 1000 L, or above 1000 L. In a preferred embodiment, a cell culture vessel is mainly composed of stainless steel, plastic, a blend of plastic materials, glass, or a combination of two or more thereof (e.g., a composite material).

As indicated above, the inner surface of the cell culture vessel of the invention bears one or more immobilized lectins.

As used herein, the term “immobilized” may be understood in the broadest sense. It may be understood as attachment of the one or more lectins to the inner surface of the cell culture vessel by any means. Immobilization may be covalently or non-covalently. It may be covalent linkage between the one or more lectins and the inner surface or may be a complex formation or may be a non-covalent interaction such as a ionic interaction, an interaction of hydrogen bonds, a hydrophobic interaction, or a combination thereof. Immobilization may be irreversibly or reversibly.

Immobilization may have any binding strength. In a preferred embodiment, immobilization is characterized in that not more than 90%, not more than 75%, not more than 50%, not more than 25%, or not more than 10%, of the respective lectin immobilized to the surface on the onset of incubating cells is washed off the surface within 1 day of incubation at 37° C., in particular when incubated in DMEM standard medium without serum.

The inner surface may be composed of any materials suitable for cell culture. Preferably, such inner surface is composed of one or more materials that may be brought into contact with cell culture medium containing the cells without harming the cells.

As used throughout the present invention, a cell culture medium may be any medium that is suitable for cultivating cells therein. A cell culture medium may be liquid or solid. In a preferred embodiment, a cell culture medium is a liquid cell culture medium. A cell culture medium may or may not comprise serum such as fetal calf serum (FCS) or bovine serum. In a preferred embodiment, a cell culture medium is a liquid cell culture medium. In a preferred embodiment, the cell culture medium is serum-free. In a preferred embodiment, the cell culture medium is free of antibiotics, in particular free of penicillins, cephalosporins, aminoglycosides, tetracyclines, macrolides and fluoroquinolones. In a preferred embodiment, the cell culture medium is serum-free and free of antibiotics.

In a preferred embodiment, the inner surface of the cell culture vessel is mainly composed of stainless steel, plastic, a blend of plastic materials, glass, or a combination of two or more thereof (e.g., as a composite material).

As used herein, plastic may be any plastic suitable for cell culture, preferably any plastic that may be brought into contact with cell culture medium containing the cells without harming the cells. In a preferred embodiment, plastic may be thermoplastic material. In a preferred embodiment, plastic may be selected from the group consisting of polystyrene, polypropylene, polycarbonate, and blends of two or more thereof.

As used herein, the term “mainly composed of” may be understood in the broadest sense as containing more than 50% by weight of the designated material. In a preferred embodiment, a component that is mainly composed of a specific material contains at least 75% by weight, at least 90% by weight, at least 95% by weight of such material or even (essentially) consists of such material.

It will be understood that not necessarily the whole inner surface of the cell culture vessel has to be coated by immobilized one or more lectins. Preferably, at least some parts which are contacted with the cell culture medium containing the cells contain immobilized one or more lectins.

In a preferred embodiment, at least 10%, at least 25%, at least 50%, or at least 75% of the inner surface which is contacted with the cell culture medium containing the cells, referred to the whole inner surface which is contacted with the cell culture medium containing the cells, bear immobilized one or more lectins.

In a preferred embodiment, at least 10%, at least 25%, at least 50%, or at least 75% of the inner surface of the cell culture vessel, referred to the whole inner surface of the cell culture vessel, bear immobilized one or more lectins.

As indicated above, the one or more lectins may be immobilized on the inner surface of the cell culture vessel by any means. In a preferred embodiment, the one or more lectins:
(a) bind to a cellulose acetate coating or cellulose acetate nanofibers immobilized on the inner surface of the cell culture vessel;
(b) form part of fusion proteins that each comprise at least one polyhistidine tag that forms a chelate complex with bivalent ions, in particular nickel or cobalt ions, presented at the inner surface, optionally via electrochemical immobilization;
(c) are conjugated to nanoparticles, in particular nanodiamonds, immobilized on the inner surface of the cell culture vessel;
(d) are conjugated to microparticles, in particular organic or inorganic microbeads, immobilized on the inner surface of the cell culture vessel;
(e) are covalently bound to one binding side of a bifunctional linker that binds to the inner surface of the cell culture vessel with the other binding site;
(f) are covalently bound to a polymeric structure, in particular a polypeptide or polysaccharide structure immobilized on the surface of the inner surface of the cell culture vessel;
(g) form part of fusion proteins that each comprise at least one protein that binds to a binding moiety, in particular a small molecule binding moiety of a weight average molecular weight of not more than 1000 Da or a polypeptide binding moiety, immobilized on the inner surface of the cell culture vessel;
(h) each comprise at least one covalently bound non-peptidic binding moiety, in particular a small molecule binding moiety of a weight average molecular weight of not more than 1000 Da, that binds to a binding moiety, in particular a small molecule binding moiety of a weight average molecular weight of not more than 1000 Da or a polypeptide binding moiety, immobilized on the inner surface of the cell culture vessel;
(i) are bound by one or more lectin-binding antibodies or lectin-binding antibody fragments; or
(j) a combination of two or more thereof.

Accordingly, the inner surface of the cell culture vessel may be coated with cellulose acetate or cellulose acetate nanofibers. Then, the one or more lectins may be added to this pretreated surface and may attach thereto. Such method usable herein with adapted proteins is described by Zhong et al., Materials Science and Engineering: C, 2015, 49:251-255.

In another preferred embodiment, the one or more lectins form part of fusion proteins that each comprise at least one polyhistidine tag that forms a chelate complex with bivalent ions, in particular nickel or cobalt ions, presented at the inner surface, optionally via electrochemical immobilization. In a preferred embodiment, a polyhistidine tag (also: histidine tag or His tag) may comprise five, six or seven consecutive histidine moieties, in particular six histidine moieties (also: hex histidine tag or His₆ tag). Suitable bivalent ions may for instance comprise transitional metal ions such as Mn²⁺, Fe²⁺, Co²⁺, Ni²⁺ and/or Cu²⁺. In a preferred embodiment, the ions in the center of the chelate complex are nickel ions (Ni²⁺). Any chelator suitable for this purpose may be used an immobilized on the inner surface of the cell culture vessel such as iminodiacetic acid, nitrilotriacetic acid, or carboxyl-methyl aspartate, or a combination thereof. A method that may be used for attaching a polyhistidine tag-(His₆− tag-) labeled protein in combination with a metal surface by an electrochemical process is described by Haruyama et al. Biomaterials, 2005, 26(24):4944-4947. This can also be used for lectin labelled with a polyhistidine tag.

A lectin may comprise one or more additional cysteine moieties such as, e.g., an N-terminal cysteine residue. This allows conjugating via further means such as, for instance, to a gold surface.

Lectins may also be immobilized on nanoparticles, in particular nanodiamonds, which may then be immobilized on the inner surface of the cell culture vessel. The use of nanodiamonds is described in Wu et al. (Angewandte Chemie International Edition, 2016, 55(23):6586-6598).

Lectins may also be immobilized on microparticles, which may then be immobilized on the inner surface of the cell culture vessel. Such microparticles may be organic or inorganic microbeads such as, e.g., silica beans, agarose beads, Sepharose beads, etc., and may optionally be (ferro) magnetic beads.

In another preferred embodiment, the one or more lectins are covalently bound to the inner surface of the cell culture vessel via a bifunctional linker that binds to the inner surface of the cell culture vessel with the other binding site. A reactive group that binds to the lectin may, for instance, be a succinimidyl ester (e.g., N-hydroxy-succinimide (NHS)), a halogen active ester, a maleimide moiety, an epoxy group, an anhydride (e.g., —CO—CO— or —COCl). Cross-linking of proteins and polysaccharide may also be achieved by addition of glutaraldehyde to a mixture.

In a further preferred embodiment, the inner surface of the cell culture vessel or parts thereof is coated with a gold layer. This may for instance be a layer of a thickness of less than 100 μm, less than 10 μm or less than 1 μm. Such layer may be prepared by any means such as, e.g., via evaporation (also: vacuum deposition) or electrochemically galvanic/electroplated). Then, the one or more lectins may be added either via a bivalent linker as described above binding to the lectin(s) or via one or more thiol groups comprised in the one or more lectins (preferably of cysteine residues). When conjugating a lectin to a gold surface of a vessel and/or a bead, the lectin may comprise one or more additional cysteine moieties such as, e.g., an N-terminal cysteine residue.

In a further preferred embodiment, the inner surface of the cell culture vessel or parts thereof is coated with a coating that comprises either functional groups to which the one or more lectins are bound directly or via a bivalent liker as described above.

In a further preferred embodiment, the inner surface of the cell culture vessel or parts thereof is coated with a coating that comprises one or more lectins, either admixed in unbound form or bound to one or more components of the coating. Such coating may, for instance, be an epoxide resin or a thermoplastic resin such as polyamide, polycarbonate, poly(methacrylate), poly(methyl methacrylate) (PMMA), a polyimine, or a blend or copolymer of two or more thereof. Optionally, the one or more lectins may be covalently bound to such polymer via amide and/or ester bonds. Preferably, the coating material is non-toxic for cells, in particular eukaryotic cells), when after it is dried or otherwise cured. Preferably, the coating material is water-soluble before curing and water-insoluble after curing. For instance, it may be an acryl polymer dispersion.

The one or more lectins may also be bound to a polymeric structure that is bound to the inner surface of the cell culture vessel. Such polymeric structure may for instance be a polysaccharide, a polypeptide or a thermoplastic material.

Lectins may also form part of fusion proteins that each comprise at least one protein that binds to a binding moiety, in particular a small molecule binding moiety of a weight average molecular weight of not more than 1000 Da or a polypeptide binding moiety, immobilized on the inner surface of the cell culture vessel. For instance, a lectin may form part of a fusion protein with streptavidin, while biotin is attached to the inner surface of the cell culture vessel. Then, streptavidin may interact with the coated biotin and thereby immobilize the lectin. A similar principle is achieved by methotrexate-dihydrofolate reductase (DHFR) binding and other binding partners.

Lectins may also each comprise at least one covalently bound non-peptidic binding moiety, in particular a small molecule binding moiety of a weight average molecular weight of not more than 1000 Da, that binds to a binding moiety, in particular a small molecule binding moiety of a weight average molecular weight of not more than 1000 Da or a polypeptide binding moiety, immobilized on the inner surface of the cell culture vessel. Such small molecule may, for instance be biotin, methotrexate and others, while the respective binding protein ma y be previously immobilized on the inner surface of the cell culture vessel.

In another preferred embodiment, lectins are bound by one or more lectin-binding antibodies or lectin-binding antibody fragments. Then, the one or more lectin-binding antibodies or lectin-binding antibody fragments may be attached to the inner surface of the cell culture vessel and may bind the one or more lectins of interest. As noted above, it will be apparent that lectins may also be fusion proteins of natural lectin sequences or saccharide-binding fragments thereof and other polypeptide sequences. Then, it will be apparent that the lectin-binding antibodies or lectin-binding antibody fragments may also directed to the fused sequence. As used in the context of the present invention, the term “antibody” may be understood in the broadest sense as any type of immunoglobulin or mutant thereof known in the art. Exemplarily, antibody invention may be an immunoglobulin A (IgA), immunoglobulin D (IgD), immunoglobulin E (IgE), immunoglobulin G (IgG), immunoglobulin M (IgM), immunoglobulin Y (IgY) or immunoglobulin W (IgW). It will be understood that the type of antibody may be altered by biotechnological means by cloning the gene encoding for the antigen-binding domains of the antibody of the present invention into a common gene construct encoding for any other antibody type. The binding between the antibody or antibody fragment and its molecular target structure (i.e., the respective lectin) typically is a non-covalent binding. Preferably, the binding affinity of the antibody to its antigen has a dissociation constant (Kd) of less than 1 μM, less than 500 nM, less than 200 nM, less than 100 nM, less than 50 nM, less than 40 nM, less than 30 nM or even less than 20 nM. As used herein, the term “antibody fragment” may be understood in the broadest sense as any fragment of an antibody that still bears binding affinity to its molecular target (i.e., the lectin). Exemplarily, the antibody fragment may be a fragment antigen binding (Fab fragment), Fc, F(ab′)2, Fab′, disulfide-linked Fv (dsFv), single chain Fv (scFv), dimers (diabodies), trimers (triabodies) or larger aggregates such as TandAbs and Flexibodies. a truncated antibody comprising one or both complementarity determining region(s) (CDR(s)), the variable fragment (Fv) of an antibody, or a cameloid species antibody. Variable domains (Fvs) are the smallest fragments with an intact antigen-binding domain consisting of one VL and one VH. As mentioned above, the term “antibody” may also include an antibody mimetic which may be understood in the broadest sense as organic compounds that, like antibodies, can specifically bind antigens and that typically have a molecular mass in a range of from approximately 3 kDa to approximately 25 kDa. Antibody mimetics may be, e.g., affibody molecules (affibodies), affilins, affitins, anticalins, avimers, DARPins, Fynomers, Kunitz domain peptides, single-domain antibodies (e.g., VHH antibodies or VNAR antibodies, nanobodies), monobodies, diabodies, triabodies, flexibodies and tandabs. The antibody mimetics may be of natural origin, of gene technologic origin and/or of synthetical origin. The antibody mimetics may also include polynucleotide-based binding units.

It will be understood that optionally also two or more of the aforementioned means for immobilization of lectins may be combined with each other.

The immobilized lectin may be the only antimicrobial component in the cell culture or may be combined with one or more further components for decreasing microorganism contamination. In particular, the immobilized lectin may be the only antimicrobial component immobilized on the inner surface of the cell culture vessel or may be combined with one or more further components immobilized on the inner surface of the cell culture vessel for decreasing microorganism contamination.

In a preferred embodiment, the inner surface of the cell culture vessel further comprises silver nanoparticles or colloidal silver.

The attachment of silver to surfaces such as polymeric surfaces may be based on nano-silver and may be conducted as described in Riau et al., Pathogens, 2019, 8(93). Colloidal silver usable for coating the inner surface of the cell culture vessel may also be conducted as described in WO 2004/017738. Silver coating of stainless steel surfaces may be conducted as described in Devasconcellos et al., Mater Sci Eng C Mater Biol Appl, 2012, 32(5): 1112-1120, and Chen et al., Surface and Coatings Technology, 2010, 204(23): 3871-3875.

In a plastic material usable as inner surface of the cell culture vessel, a silver component may be included as described in WO 2014/138885. Furthermore, silver nitrate nanoparticles may be mixed with lectin as described in Anjana and Anitha, Int. J. Curr. Sci., 2012, 17-23, and subsequently attached to the inner surface of the cell culture vessel.

As indicated above, the cell culture vessel of the present invention is particularly suitable for cultivating cells and is thereby able to reduce contamination with microorganisms.

Accordingly, a further aspect of the present invention relates to a method for cultivating cells comprising the following steps:
(i) providing cells in a cell culture medium;
(ii) contacting the cell culture medium comprising the cells with a surface on which one or more lectins suitable for binding to one or more species of microorganisms are immobilized; and
(iii) cultivating the cells in the cell culture medium in contact with the surface.

It will be understood that all definitions and preferred embodiments made in the context of the cell culture vessel herein may mutatis mutandis also refer to the method for cultivating cells of the present invention.

Accordingly, the present invention also relates to a method for cultivating cells comprising cultivating cells of interest in cell culture medium.

The method for cultivating cells may be usable for culturing any cells. In a preferred embodiment, it is for culturing eukaryotic cells. In a preferred embodiment, it is for culturing human, animal, fungal or plant cells. In a preferred embodiment, it is for culturing human or animal cells, in particular human or other mammalian cells. Herein, the designation of cells may be understood in a broad sense as indicating the genetic origin thereof, while the cells may optionally also be obtained from a cell bank and/or may be stored.

In a preferred embodiment, the surface on which one or more lectins suitable for binding to one or more species of microorganisms are immobilized is the inner surface of a cell culture vessel of the present invention.

In another preferred embodiment, the surface on which one or more lectins suitable for binding to one or more species of microorganisms are immobilized is the outer surface of beads contacted with the cell culture medium.

Such beads may be attached to the inner surface of a cell culture vessel and/or may be suspended in the cell culture medium. In a preferred embodiment, the beads are suspended in the cell culture medium. Such beads may have any size range. In a preferred embodiment, the beads have a mass average diameter of 1 μm to 5 mm, as determinable by sieving. In a preferred embodiment, the beads have a mass average diameter of 5 μm to 2 mm, of 1 μm to 1 mm, of 10 μm to 500 μm, of 20 μm to 200 μm, or of 50 μm to 100 μm, as determinable by sieving. The beads may be composed of any material. In a preferred embodiment, the outer surface of the beads is suitable for cells. In a preferred embodiment, the beads are (ferro) magnetic beads and the method further comprises the step of removing the beads by applying a magnetic field. Such (ferro) magnetic beads are obtainable from commercial sources such as Miltenyi Biotec B.V. & Co. KG (Bergisch Gladbach, Germany).

The person skilled in the art will know routine techniques for cultivating cells. For instance, cells of animal origin (animal cells) may be cultivated in a liquid medium such as DMEM/F-12 at a temperature of 35-38° C. (preferably 37° C.) and at 5% CO₂. The person skilled in the art will be able to adapt cultivation conditions for any cell type investigated.

As indicated above, the method for cultivating cells of the present invention is suitable for having reduced content of microorganisms to which the lectins bind. In a preferred embodiment, the method is for cultivating cells in an environment having reduced content of Mycoplasma species in the cell culture medium. It will be understood that the person skilled in the art will choose the respective lectins.

As used throughout the present invention, a cell culture medium may be any medium that is suitable for cultivating cells therein. In a preferred embodiment, the cell culture medium is serum-free. In a preferred embodiment, the cell culture medium is free of antibiotics, in particular free of penicillins, cephalosporins, aminoglycosides, tetracyclines, macrolides and fluoroquinolones. In a preferred embodiment, the cell culture medium is serum-free and free of antibiotics.

The cell mass cultivated may be used for any purpose. In a preferred embodiment, the method is for providing a cell mass comestible by mammalian consumers. In this context, the method preferably comprises the step of separating the cultivated cells from the antimicrobial surface.

Alternatively or additionally, the cell culture method may be used for the homologous or heterologous expression of polypeptides (e.g., monoclonal or polyclonal antibodies or antibody fragments, of hormones, or other proteins), small-molecular sugars, polysaccharides, lipids, or vitamins. Alternatively or additionally, the cell culture method may be used for investigating the effects of chemical agents, hormones, medicaments, pharmaceutical candidates, toxins and mixtures thereof, of cell culture media, effects of pathogens such a viruses, bacteria and protozoa, of temperature, pH, and/or irradiations. Alternatively or additionally, the cell culture method may be used for investigating intracellular purposes, cell differentiation, de-differentiation, mutagenesis, neoplastic progression of cells. The interaction of different cell types and even cells of different species are observable in such cell culture method.

As indicated above, a special technical feature is the use of immobilized lectins for reducing microorganism contamination.

Thus, a further aspect relates to the use of a lectin suitable for binding to one or more species of microorganisms, which is immobilized on a solid surface, for reducing the microorganism contamination in a cell culture contacted with the surface.

It will be understood that all definitions and preferred embodiments made in the context of the cell culture vessel and method herein may mutatis mutandis also refer to the use of the present invention.

The present invention also refers to the use of a lectin suitable for binding to one or more species of microorganisms for preparing an antimicrobial surface, wherein the lectin is conjugated to the surface, for reducing the microorganism contamination in a cell culture contacted with the antimicrobial surface.

Moreover, an aspect of the present invention relates to a method for preparing an antimicrobial surface suitable for cell culture, comprising the step of conjugating one or more lectins each suitable for binding to one or more species of microorganisms to the surface.

It will be understood that all definitions and preferred embodiments made in the context of the cell culture vessel, method for cultivating cells and use herein may mutatis mutandis also refer to the method for preparing an antimicrobial surface of the present invention.

The following examples illustrate and exemplify the invention further without limiting its scope. The scope of the invention is defined by the claims.

### EXAMPLES

### Example 1—Coating a Cell Culture Vessel Via a Gold/Lectin Surface

### Preparation of a Cell Culture Vessel Having a Gold/Lectin Surface

A cell culture vessel of stainless steel having a gold-coated inner layer is provided by a commercial supplier or is prepared by vacuum deposition of a thin gold layer or via applying a gold salt (e.g., AuCl) to the surface, optionally accompanied via an electrical current, and depositing elemental gold via a redox reaction. One or more lectins of interest (e.g., surfactant protein A (SP-A)) comprising at least one cysteine moiety bearing a thiol group which is dissolved in a buffer (e.g., a PBS buffer) are added to the surface and incubated, optionally until the fluid is evaporated.

### Use of a Cell Culture Vessel Having a Gold/Lectin Surface

The cell culture vessel having a gold/lectin surface may be used for cell culture purposes in a common way. The number of microbes is reduced by interaction with the one or more lectins (e.g., surfactant protein A (SP-A)) of the vessel's inner surface contacted with the cell culture medium.

### Example 2—Coating a Cell Culture Vessel Via a Lectin-Containing Coating

Preparation of a cell culture vessel having a lectin-containing coating A non-toxic coating material (e.g., an acryl polymer dispersion or epoxy resin), is admixed with one or more lectins of interest (e.g., surfactant protein A (SP-A)). The material is applied to the inner surface of the cell culture vessel (which may, e.g., be of stainless steel) and cured (e.g., by drying). Thereby, a water-insoluble coating may be obtained. This may optionally be washed.

### Use of a Cell Culture Vessel Having a Lectin-Containing Coating

The cell culture vessel having a coating containing one or more lectins may be used for cell culture purposes in a common way. The number of microbes is reduced by interaction with the one or more lectins (e.g., surfactant protein A (SP-A)) of the vessel's inner surface contacted with the cell culture medium.

### Example 3—Magnetic Bead Coated with a Lectin

Preparation of Magnetic Bead Coated with a Lectin

A solution of the one or more lectins (e.g., surfactant protein A (SP-A)) of interest is prepared. Magnetic beads with activated functional groups on their surface (e.g., commercially available MACSflex™ MicroBeads (MACSflex™ Starting Kit) from Miltenyi Biotec B.V. & Co. KG (Bergisch Gladbach, Germany) having N-hydroxy-succinimide (NHS) ester groups on their surface) are admixed with the solution of the one or more lectins. Thereby, a reaction of the surface of the magnetic beads takes place with the one or more lectins.

Optional Purification of Magnetic Bead Coated with a Lectin

The magnetic beads coated with the one or more lectins (e.g., surfactant protein A (SP-A)) are isolated from the dispersion via applying a magnetic force (e.g, via a μMACS™ Separator from Miltenyi Biotec B.V. & Co. KG (Bergisch Gladbach, Germany)). This allows several washing steps with buffer such as PBS.

Use of Magnetic Bead Coated with a Lectin

The magnetic beads coated with the one or more lectins (e.g., surfactant protein A (SP-A)) are added to a cell culture, incubated for typically at least few hours and are subsequently removed from the cell culture or a product obtained therefrom via applying a magnetic force.

1-15. (canceled)

16. A cell culture vessel comprising an inner surface, wherein one or more lectins suitable for binding to one or more species of microorganisms are immobilized on the inner surface.

17. The cell culture vessel of claim 16, wherein the one or more lectins are selected from the group consisting of: C-type lectins, a fragment suitable for binding to one or more species of microorganisms thereof, and a fusion protein comprising a C-type lectin or a fragment suitable for binding to one or more species of microorganisms thereof.

18. The cell culture vessel of claim 16, wherein the one or more lectins are selected from the group consisting of: collectins, a fragment suitable for binding to one or more species of microorganisms thereof, and a fusion protein comprising a collectin or a fragment suitable for binding to one or more species of microorganisms thereof.

19. The cell culture vessel of claim 16, wherein the one or more lectins are selected from the group consisting of: surfactant protein A, surfactant protein D, mannose-binding lectin, collectin liver 1, collectin placenta 1, collectin kidney 1, conglutinin, collectin of 43 kDa, collectin of 46 kDa, a fragment suitable for binding to one or more species of microorganisms thereof, and a fusion protein comprising at least one thereof.

20. The cell culture vessel of claim 16, wherein the one or more lectins comprise surfactant protein A or a fusion protein comprising surfactant protein A.

21. The cell culture vessel of claim 16, wherein the one or more species of microorganisms are mollicutes.

22. The cell culture vessel of claim 16, wherein the inner surface of the cell culture vessel mainly comprises stainless steel, plastic, a blend of plastic materials, glass, or a combination of two or more thereof.

23. The cell culture vessel of claim 16, wherein the one or more lectins:
(a) bind to a cellulose acetate coating or cellulose acetate nanofibers immobilized on the inner surface of the cell culture vessel;
(b) form part of fusion proteins, wherein each fusion protein comprises at least one polyhistidine tag, and wherein the at least one polyhistidine tag forms a chelate complex with bivalent ions presented at the inner surface, optionally via electrochemical immobilization;
(c) are conjugated to nanoparticles immobilized on the inner surface of the cell culture vessel;
(d) are conjugated to microparticles immobilized on the inner surface of the cell culture vessel;
(e) are covalently bound to one binding side of a bifunctional linker, wherein the one binding side of the bifunctional linker binds to the inner surface of the cell culture vessel with another binding side of the bifunctional linker;
(f) are covalently bound to a polymeric structure immobilized on a surface of the inner surface of the cell culture vessel;
(g) form part of fusion proteins, wherein each fusion protein comprises at least one protein, and wherein the at least one protein binds to a binding moiety immobilized on the inner surface of the cell culture vessel;
(h) each comprises at least one covalently bound non-peptidic binding moiety, wherein the at least one covalently bound non-peptidic binding moiety binds to a binding moiety immobilized on the inner surface of the cell culture vessel;
(i) are bound by one or more lectin-binding antibodies or lectin-binding antibody fragments; or
(j) a combination of two or more thereof.

24. The cell culture vessel of claim 16, wherein the inner surface of the cell culture vessel comprises silver nanoparticles or colloidal silver.

25. A method for cultivating cells comprising:
(i) providing cells in a cell culture medium;
(ii) contacting the cell culture medium comprising the cells with a surface on which one or more lectins suitable for binding to one or more species of microorganisms are immobilized; and
(iii) cultivating the cells in the cell culture medium in contact with the surface.

26. The method of claim 25, wherein the surface is an inner surface of a cell culture vessel, and wherein the one or more lectins suitable for binding to one or more species of microorganisms are immobilized on the inner surface.

27. The method of claim 25, wherein the surface is an outer surface of beads contacted with the cell culture medium.

28. The method of claim 27, wherein the beads are suspended in the cell culture medium.

29. The method of claim 27, wherein the beads have a mass average diameter of 1 μm to 5 mm, and wherein the diameter of the beads is determined by sieving.

30. The method of claim 27, wherein the beads are (ferro) magnetic beads, and wherein the method further comprises the step of removing the beads by applying a magnetic field.

31. The method of claim 27, wherein the cells are cultivated in an environment having reduced content of Mycoplasma species in the cell culture medium.

32. The method of claim 27, wherein the cell culture medium is free of penicillins, cephalosporins, aminoglycosides, tetracyclines, macrolides, and fluoroquinolones.

33. The method of claim 27, wherein the method further comprises the step of providing a cell mass comestible by mammalian consumers.

34. The method of claim 33, wherein the method further comprises the step of separating the cultivated cells from the surface.

35. A method for reducing a microorganism contamination in a cell culture contacted with a solid surface, comprising the step of immobilizing a lectin suitable for binding to one or more species of microorganisms on the solid surface.
